(19) United States
(12) Patent Application Publication
(10) Pub. No.: US 2014/0294920 A1
(43) Pub. Date: Oct. 2, 2014

(54) TRANSDERMAL PROPRANOLOL PATCH FOR TREATMENT OF MALIGNANT MELANOMA

(71) Applicant: Nometics Inc., Kingston (CA)

(72) Inventor: David J. Enscore, Alpharetta, GA (US)

(21) Appl. No.: 14/280,126

(22) Filed: May 16, 2014

Related U.S. Application Data
(63) Continuation of application No. PCT/IB2012/002727, filed on Nov. 16, 2012.
(60) Provisional application No. 61/585,084, filed on Jan. 10, 2012.
(60) Provisional application No. 61/560,581, filed on Nov. 16, 2011.
(60) Provisional application No. 61/560,582, filed on Nov. 16, 2011.
(60) Provisional application No. 61/585,085, filed on Jan. 10, 2012.

(51) Int. Cl. A61K 31/138; A61K 8/02; A61K 8/41; A61Q 19/02; A61K 9/70

(52) U.S. Cl. CPC A61K 31/138; A61Q 19/02; A61K 9/7084; A61K 8/41; A61K 8/027
USPC 424/449; 514/652; 424/62

(57) ABSTRACT

A transdermal d propranolol or racemic propranolol patch for use as an adjunct to surgery in the treatment of malignant melanoma and/or tattoo related applications are herein disclosed.

### RELATED APPLICATIONS

This application claims priority to U.S. provisional application Nos. 61/560,581, filed on Nov. 16, 2011; 61/560,582, filed on Nov. 16, 2011; 61/585,085, filed on Jan. 10, 2012; and 61/585,084, filed on Jan. 10, 2012, all of which are hereby incorporated by reference in their entirety.

### BACKGROUND

Despite improvements in surgical and radiological procedures and an increasingly large arsenal of drugs for treating cancer, cures for many melanomas remain elusive. Malignant melanoma is an excellent example of a virulent cancer that is treatable by surgical removal with a high rate of success if detected before metastasis. After metastasis, however, the five year mortality rate is very high, even when using available chemotherapy and radiotherapy techniques.

Accordingly, there remains a need for an effective melanoma treatment.

### SUMMARY OF THE INVENTION

In an aspect, provided herein is a method of treating malignant melanoma in a subject that includes administering to the subject a transdermal patch comprising an effective amount of d propranolol or a pharmaceutically acceptable salt thereof.

In an aspect, a method of treating malignant melanoma in a subject is disclosed that includes administering to the subject a transdermal patch comprising an effective amount of racemic propranolol or a pharmaceutically acceptable salt thereof.

Embodiments of a transdermal d propranolol and/or racemic propranolol patch for use as an adjunct to surgery in the treatment of malignant melanoma are herein disclosed.

In an embodiment, the method of treatment uses a transdermal patch that has an adhesive layer, a system backing, a release liner, and a membrane. In an embodiment, the method uses a patch that has a system backing that is a single polymer layer, wherein the system backing is selected from polyethylene, polypropylene, polyethylene terephthalate, polystyrene, ionomer polyurethane, acrylonitrile, polycarbonate, acrylates, acrylonitrile-methyl acrylate copolymer, an ethylene/vinyl acetate heat seal layer, and a layer of aluminum. In an embodiment, the method uses a patch that has a system backing that is a multilaminate film, wherein the system backing is selected from polyethylene, polypropylene, polyethylene terephthalate, polystyrene, ionomer polyurethane, acrylonitrile, polycarbonate, acrylates, acrylonitrile-methyl acrylate copolymer, an ethylene/vinyl acetate heat seal layer, and a layer of aluminum. In an embodiment, the system backing is a release liner selected from kraft paper, polypropylene, polystyrene, polyethylene terephthalate, acrylonitrile, polycarbonate, acrylates, and acrylonitrile-methyl acrylate copolymer. In an embodiment, the release liner is a release agent selected from silicone polymer and fluorocarbon diacrylate polymer. In another embodiment, the d propranolol and/or racemic propranolol is contained in a single adhesive layer between the system backing and the release liner. In yet another embodiment, the d propranolol or racemic propranolol is contained in a solid layer between the system backing and the release liner. In another embodiment, the d propranolol or racemic propranolol is contained in a liquid layer sealed between the system backing and the membrane. In another embodiment, the d propranolol or racemic propranolol is contained in a colloidal suspension or gel layer between the system backing and said release liner. In another embodiment, the d propranolol or racemic propranolol is contained in a matrix layer that is non- adhesive, and the transdermal patch further contains an overlay adhesive. In another embodiment, the d propranolol or racemic propranolol is administered to the skin of the subject through an excipient selected from ointments, liniments, pastes, films, hydrogels, liposomes, transfersome vesicles, creams, lotions, lip balms, shampoos, dermal patches, transdermal sprays and jet injectors.

In an embodiment of the methods provided herein, the subject is human.

In an aspect, a method of bleaching skin in a subject is disclosed that includes administering to the subject an effective amount of d propranolol and/or racemic propranolol.

In another aspect, a method of removing pigment from the skin in a subject is disclosed that includes administering to the subject an effective amount of d propranolol and/or racemic propranolol.

In an aspect, a method of tattooing the skin in a subject is disclosed that includes administering to the subject an effective amount of d propranolol and/or racemic propranolol.

### BRIEF DESCRIPTION OF THE DRAWINGS

FIG. 1 depicts a cross-sectional view of a matrix transdermal patch provided herein.

FIG. 2 depicts a cross-sectional view of a multilaminate transdermal patch provided herein with a solid drug reservoir.

FIG. 3 depicts a cross-sectional view of a multilaminate transdermal patch provided herein with a liquid drug reservoir.

### DETAILED DESCRIPTION OF THE INVENTION

Provided herein is a transdermal patch comprising d propranolol or racemic propranolol that can be used for the treatment of melanoma.

Transdermal delivery of d propranolol or racemic propranolol is proportional to the transdermal drug flux. The associated skin site exhibits a reduction in its response to ultraviolet radiation, varying from reduced tanning following a relatively mild irritation response to elimination of tanning following a strong irritation response. Destruction of melanocyte cells in the skin under the application site is the cause of the tanning response effect. Following strong topical irritation from d propranolol and/or racemic propranolol, the melanocytes in the underlying skin are completely eliminated.

Transdermal d propranolol or racemic propranolol transdermal delivery is therefore useful as an adjunct to surgical excision of the tumor in the treatment of pre-metastatic malignant melanoma, both immediately following surgery and for prophylaxis of disease recurrence over time.

One of the differences between the racemic propranolol and the d propranolol effects in a subject is that the racemic propranolol may cause a transient reduction in blood pressure in the subject whereas this response is essentially avoided by the use of an essentially enantiopure d propranolol composition.

High transdermal propranolol and/or racemic propranolol flux is desired to maximize the irritation responses and attendant melanocyte destruction. The transdermal propranolol and/or racemic propranolol flux can be controlled through selection of the adhesive layer materials and the loading of drug in the adhesive layer or by inclusion of a membrane between the drug reservoir layer and the adhesive layer. Higher transdermal drug flux can be achieved by incorporation of a drug permeation enhancer into the formulation.

The system backing of the present invention may be a single polymer layer or a multilaminate film. Single layer polymer films that may be employed include, without limitation, polyethylene terephthalate (PET), polyethylene, polypropylene, polystyrene, ionomer, polyurethane, acrylonitrile, polycarbonate, acrylates, and acrylonitrile-methyl acrylate copolymer. The backing film may have additional layers such as a polyethylene or ethylene/vinyl acetate heat seal layer or a layer of aluminum to impart opacity.

The realease liner of the present invention may be selected, without limitation, from kraft paper, polyethylene terephthalate, polypropylene, polystyrene, acrylonitrile, polycarbonate, acrylates, and acrylonitrile-methyl acrylate copolymer. The release liner also will contain a layer of release agent such as silicone polymer, fluorocarbon diacrylate polymer, or any other agent commonly employed to provide adhesive release.

The adhesive of the present invention may be selected, without limitation, from polyisobutylene, acrylate polymers, styrene isoprene styrene copolymer (SIS), and silicone adhesive polymers. The polyisobutylene adhesive may contain a plasticizer such as mineral oil and the SIS adhesive may contain a tackifier such as an aliphatic resin ester.

In an embodiment, permeation enhancing agents of the present disclosure may be selected, without limitation, from dimethyl sulfoxide, n-decyl methyl sulfoxide, glycerol monolaurate, isopropyl myristate, and sucrose monococoate.

Excipients and other delivery vessels for d propranolol and/or racemic propranolol may be selected from the group consisting of ointments, liniments, pastes, films, hydrogels, liposomes, transfersome vesicles, creams, lotions, lip balms, shampoos, dermal patches, transdermal sprays and jet injectors.

In an embodiment, racemic propranolol and/or d-propranolol can also be used for tattoo applications. Without being bound by theory, as discussed above, the d propranolol and/or racemic propranolol can be used for the killing/suppression of melanocytes with a consequent “bleaching” of the skin. As such, creating a white background on skin could be used to enhance color presentation of a traditional tattoo on the skin. For subjects with darker skin, such a technique could be used to create an outline for a traditional, permanent tattoo, or used itself as a temporary tattoo. The d-propranolol and/or racemic propranolol can be delivered through a transdermal patch, a dermal patch, a traditional tattoo injector, needle, creams, lotions or other vessels.

Embodiments of transdermal skin patches disclosed herein are useful for delivering an effective dose of d propranolol or racemic propranolol to the skin of a subject.

As used herein, the term “pharmaceutically acceptable salts” refers to the nontoxic acid or alkaline earth metal salts of the drugs used in embodiments of transdermal patches disclosed herein. Representative salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2-hydroxyethanesulfonate, lactate, maleate, methanesulfonate, nicotinate, 2-naphth-alenesulfonate, oxalate, pamoate, pectinate, persulfate, 3-phenylproionate, phosphate, picrate, pivalate, propionate, succinate, sulfate, tartrate, thiocyanate, p-toluenesulfonate, and undecanoate.

Examples of acids that may be employed to form pharmaceutically acceptable acid addition salts include such inorganic acids as hydrochloric acid, hydrobromic acid, nitric acid, sulfuric acid and phosphoric acid and such organic acids as formic acid, acetic acid, trifluoroacetic acid, fumaric acid, tartaric acid, oxalic acid, maleic acid, methanesulfonic acid, succinic acid, malic acid, methanesulfonic acid, benzenesulfonic acid, and p-toluenesulfonic acid, citric acid, and acidic amino acids such as aspartic acid and glutamic acid.

The term “treat” is used herein to mean to relieve, reduce or alleviate, at least one symptom of a disease or condition in a subject. The term “treat” also denotes, to arrest, delay the onset (i.e., the period prior to clinical manifestation of a disease or symptom of a disease) and/or reduce the risk of developing or worsening a symptom of a disease or condition.

The term “subject” is intended to include animals in addition to humans. Examples of subjects include mammals, e.g., humans, dogs, cows, horses, pigs, sheep, goats, cats, mice, rabbits, rats, and transgenic non-human animals.

The term “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

An “effective amount” is an amount sufficient to provide an observable improvement over the baseline clinically observable signs and symptoms of the disease or symptom treated.

In one embodiment, a transdermal d propranolol and/or racemic propranolol patch is disclosed that contains polyethylene terephthalate (PET). PET forms a barrier polymer film and is used as the system backing and/or release liner components of many transdermal patches,

Transdermal d propranolol and/or racemic propranolol patches disclosed herein can be constructed using designs and methods of making transdermal patches well known in the art. The specific embodiment of the d propranolol and/or racemic propranolol transdermal patch, while important for other performance attributes, does not impact the transdermal d propranolol and/or racemic propranolol patches disclosed herein.

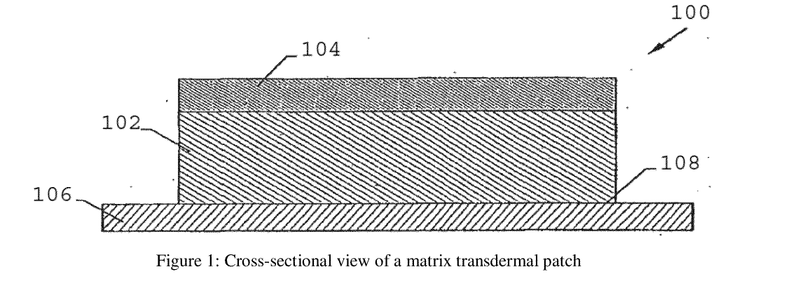

In an embodiment, transdermal drug patches of the present disclosure may be of a matrix type where the drug is contained in a single adhesive layer between the system backing and the release liner. FIG. 1 depicts an embodiment of a transdermal drug patch 100. Transdermal drug patch 100 contains a drug contained in the adhesive layer 102 which is located between the system backing 104, and the release liner 106. In an embodiment of matrix design transdermal drug patch 100, the drug contained in adhesive layer 102 is d propranolol and/or racemic propranolol. If the propranolol is enantiomerically pure, such as d propranolol, stereoisomers, and racemates are generally not part of the drug composition.

In one embodiment, a dense membrane layer 108 may be used to form an enclosure to release liner 106 and/or (not depicted in FIG. 1) to system backing 104.

In an embodiment, system backing 104 may be a single polymer layer or a multilaminate film including, without limitation, polyethylene, polypropylene, polystyrene, ionomer polyurethane, acrylonitrile, polycarbonate, acrylates, and acrylonitrile-methyl acrylate copolymer. In an embodiment, system backing 104 may contain additional layers such as polyethylene, an ethylene/vinyl acetate heat seal layer or a layer of aluminum.

In an embodiment, release liner 106 may be selected, without limitation, from kraft paper, polypropylene, polystyrene, acrylonitrile, polycarbonate, acrylates, and acrylonitrile- methyl acrylate copolymer. In another embodiment, release liner 106 may contain a layer of release agent such as silicone polymer, fluorocarbon diacrylate polymer, or any other agent commonly employed to provide adhesive release.

In an embodiment, transdermal d propranolol and/or racemic propranolol patch 100 is composed of three functional layers. With reference to FIG. 1, and proceeding from the top, there is system backing layer 104, adhesive layer 102 containing d propranolol and/or racemic propranolol, and release liner 106. System backing 104 serves to prevent the drug from escaping from the side of the patch distal to the skin and from preventing adhesive layer 102 from adhering to clothing. Adhesive layer 102 contains the d propranolol and/or racemic propranolol to be delivered and serves to adhere the patch to the skin. Release liner 106 protects adhesive layer 102 prior to use and is removed immediately prior to patch application.

In another embodiment, the transdermal drug patches disclosed herein use matrix type design wherein the drug containing layer is a non-adhesive and the patch is held in place with an overlay adhesive.

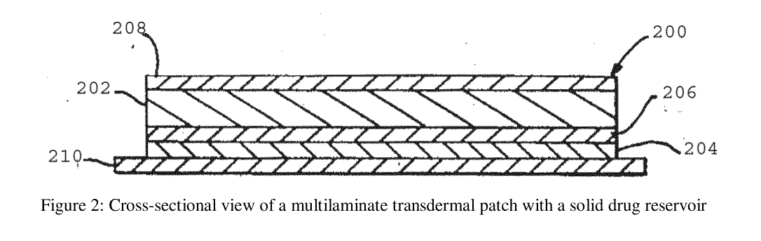

In another embodiment, transdermal drug patches of the present disclosure may be of a multilaminate design wherein the drug is held in a reservoir separate from the adhesive layer. FIG. 2 depicts and embodiment of a multilaminate design transdermal drug patch 200. Multilaminate design transdermal drug patch 200 contains a drug in a solid reservoir layer 202, which is separated from an adhesive layer 204 by membrane 206. Solid reservoir layer 202 and adhesive layer 204 are located between a system backing 208 and a release liner 210. In an embodiment, membrane 206 can be porous or dense and can provide control of the rate of drug delivery from solid reservoir layer 202 to a patient's skin. In an embodiment, adhesive layer 204 can provide control of the rate of drug delivery to a patient's skin from solid reservoir layer 202 with or without membrane 206 in between solid reservoir layer 202 and adhesive layer 204. In an embodiment of multilaminate design transdermal drug patch 200, the drug contained in solid reservoir layer 202 is d propranolol and/or racemic propranolol, optionally including stereoisomers, tautomers, racemates, solvates, metabolites, and pharmaceutically acceptable salts thereof. In another embodiment, a drug may be contained in adhesive layer 204. In an embodiment the drug contained in adhesive layer 204 is d propranolol and/or racemic propranolol, optionally including stereoisomers, tautomers, racemates, solvates, metabolites, and pharmaceutically acceptable salts thereof.

In an embodiment, system backing 208 may be a single polymer layer or a multilaminate film including, without limitation, polyethylene, polypropylene, polystyrene, ionomer polyurethane, acrylonitrile, polycarbonate, acrylates, and acrylonitrile-methyl acrylate copolymer. In an embodiment, system backing 208 may contain additional layers such as polyethylene, an ethylene/vinyl acetate heat seal layer or a layer of aluminum.

In an embodiment, release liner 210 may be selected, without limitation, from kraft paper, polypropylene, polystyrene, acrylonitrile, polycarbonate, acrylates, and acrylonitrile- methyl acrylate copolymer. In another embodiment, release liner 210 may contain a layer of release agent such as silicone polymer, fluorocarbon diacrylate polymer, or any other agent commonly employed to provide adhesive release.

In an embodiment, a transdermal d propranolol and/or racemic propranolol patch may be created as depicted in FIG. 2 wherein system backing 208 is a film of biaxially oriented polypropylene with a layer of vapor deposited aluminum on the side distal to the patch covered by a 1 layer of pigmented polyethylene. In an embodiment, solid drug reservoir 202 has a d propranolol and/or racemic propranolol base with a colloidal silicon dioxide, mineral oil, and polyisobutylene. In an embodiment, membrane 206 is rate controlling and is a microporous polypropylene film filled with mineral oil. In an embodiment, adhesive layer 204 is contains a d propranolol and/or racemic propranolol base with colloidal silicon dioxide, mineral oil, and polyisobutylene. In an embodiment, release liner 210 is a film of biaxially oriented polypropylene containing a layer of fluorocarbon diacrylate polymer release agent.

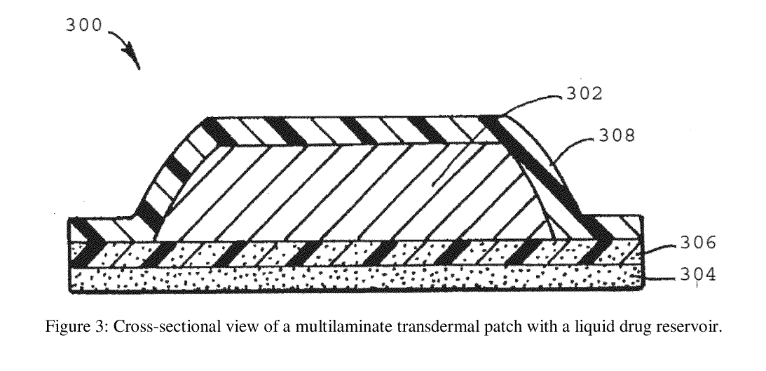

In another embodiment, transdermal drug patches of the present disclosure may be of a multilaminate design wherein the drug is held in a liquid reservoir separate from the adhesive layer. FIG. 3 depicts an embodiment of a multilaminate design transdermal drug patch 300. Multilaminate design transdermal drug patch 300 contains a drug in a liquid reservoir layer 302, which is separated from an adhesive layer 304 by membrane 306. Liquid reservoir layer 302 and membrane 306 are located between a system backing 308 and a release liner (not depicted). In an embodiment, membrane 306 can be porous or dense and can provide control of the rate of drug delivery from liquid reservoir layer 302 to a patient's skin. In an embodiment, adhesive layer 304 can provide control of the rate of drug delivery to a patient's skin from liquid reservoir layer 302 with or without membrane 306 in between liquid reservoir layer 302 and adhesive layer 304. In an embodiment, liquid reservoir layer 302 is contained by sealing the edges (with a heat seal layer, for example) of membrane 306 and system backing 308. Note that in this embodiment, the backing must have a heat seal layer

In an embodiment of multilaminate design transdermal drug patch 300, the drug contained in liquid reservoir layer 302 is a solution or suspension of d propranolol and/or racemic propranolol optionally including stereoisomers, tautomers, racemates, solvates, metabolites, and pharmaceutically acceptable salts thereof. In another embodiment, a drug may be contained in adhesive layer 304. In an embodiment the drug contained in adhesive layer 304 is d propranolol and/or racemic propranolol, optionally including stereoisomers, tautomers, racemates, solvates, metabolites, and pharmaceutically acceptable salts thereof. In an embodiment, system backing 308 may be a single polymer layer or a multilaminate film including, without limitation, polyethylene, polypropylene, polystyrene, ionomer polyurethane, acrylonitrile, polycarbonate, acrylates, and acrylonitrile-methyl acrylate copolymer. In an embodiment, system backing 308 may contain additional layers such as polyethylene, an ethylene/vinyl acetate heat seal layer or a layer of aluminum. In an embodiment, the release liner of multilaminate design transdermal drug patch 300 may be selected, without limitation, from kraft paper, polypropylene, polystyrene, acrylonitrile, polycarbonate, acrylates, and acrylonitrile-methyl acrylate copolymer. In another embodiment, the release liner of multilaminate design transdermal drug patch 300 may contain a layer of release agent such as silicone polymer, fluorocarbon diacrylate polymer, or any other agent commonly employed to provide adhesive release.

### Example

An example of a transdermal d propranolol and/or racemic propranolol patches disclosed herein contains the following: an adhesive layer that has 10 weight percent d propranolol and/or racemic propranolol base and 5 weight percent n-decyl methyl sulfoxide dispersed in a 5 mil thick layer of amine resistant silicone adhesive (BioPSA, Dow Corning); a system backing is a PET/EVA multilaminate film (ScotchPAK 9732, 3M). The release liner is a PET film with a fluorocarbon diacrylate release coating Polyester (ScotchPak 1022, 3M).

The above examples, embodiments, definitions and explanations should not be taken as limiting the full metes and bounds of the invention.

1. A method of treating malignant melanoma in a subject comprising administering to said subject a transdermal patch comprising an effective amount of d-propranolol, or a pharmaceutically acceptable salt thereof.

2. A method of treating malignant melanoma in a subject comprising administering to said subject a transdermal patch comprising an effective amount of racemic propranolol, or a pharmaceutically acceptable salt thereof.

3. A method of bleaching skin in a subject comprising administering to said subject a transdermal patch comprising an effective amount of d-propranolol, or a pharmaceutically acceptable salt thereof.

4. A method of removing pigment in the skin of a subject comprising administering to said subject a transdermal patch comprising an effective amount of d-propranolol, or a pharmaceutically acceptable salt thereof.

5. A method of tattooing the skin in a subject comprising administering to said subject a transdermal patch comprising an effective amount of d-propranolol, or a pharmaceutically acceptable salt thereof.

6. The method of any of claims 1 through 5, wherein said transdermal patch comprises an adhesive layer, a system backing, a release liner, and a membrane.

7. The method of any of claims 1 through 5, said system backing comprising a single polymer layer or a multilaminate film, said system backing is selected from the group consisting essentially of polyethylene, polypropylene, polyethylene terephthalate, polystyrene, ionomer polyurethane, acrylonitrile, polycarbonate, acrylates, acrylonitrile-methyl acrylate copolymer, an ethylene/vinyl acetate heat seal layer, and a layer of aluminum.

8. The method of any of claims 1 through 5, said system backing comprising a release liner selected from the group consisting essentially of kraft paper, polypropylene, polystyrene, polyethylene terephthalate, acrylonitrile, polycarbonate, acrylates, and acrylonitrile-methyl acrylate copolymer.

9. The method of any of claims 1 through 5, said release liner comprising a release agent selected from the group consisting essentially of silicone polymer and fluorocarbon diacrylate polymer.

10. The method of any of claims 1 through 5, wherein said d-propranolol or racemic propranolol is contained in a single adhesive layer between said system backing and said release liner.

11. The method of any of claims 1 through 5, wherein said d-propranolol or racemic propranolol is contained in a liquid layer sealed between said system backing and a membrane.

12. The method of any of claims 1 through 5, wherein said d-propranolol or racemic propranolol is contained in a colloidal suspension or gel layer between said system backing and said release liner.

13. The method of any of claims 1 through 5, wherein said d-propranolol or racemic propranolol is in a matrix layer that is non-adhesive, wherein said transdermal patch further comprises an overlay adhesive.

14. The method of claim 4, wherein said pigment removed is a component of a tattoo.

15. The method of any of claims 1 through 14, wherein the subject is human.

16. A method of treating malignant melanoma in a subject comprising administering to said subject an effective amount of d-propranolol, or a pharmaceutically acceptable salt thereof.

17. A method of treating malignant melanoma in a subject comprising administering to said subject an effective amount of racemic propranolol, or a pharmaceutically acceptable salt thereof.

18. A method of bleaching skin in a subject comprising administering to said subject an effective amount of d-propranolol, or a pharmaceutically acceptable salt thereof.

19. A method of removing pigment in the skin of a subject comprising administering to said subject an effective amount of d-propranolol, or a pharmaceutically acceptable salt thereof.

20. A method of tattooing the skin in a subject comprising administering to said subject an effective amount of d-propranolol, or a pharmaceutically acceptable salt thereof.

21. The method of any of claims 16 through 20 wherein said d-propranolol or racemic propranolol is administered to said skin of said subject through an excipient or delivery tool selected from the group consisting of ointments, liniments, pastes, films, hydrogels, liposomes, transfersome vesicles, creams, lotions, lip balms, shampoos, dermal patches, transdermal sprays and jet injectors.

22. The method of any of claims 16 through 21 wherein the subject is human.
